(19) United States
(12) Patent Application Publication
(10) Pub. No.: US 2013/0282046 A1
(43) Pub. Date: Oct. 24, 2013

(54) Adjustable garment for treatment of bodily limb pathology

(71) Applicant: Sundaram Ravikumar, Briar Cliff Manor, NY (US)

(72) Inventor: Sundaram Ravikumar, Briar Cliff Manor, NY (US)

(21) Appl. No.: 13/454,630

(22) Filed: Apr. 24, 2012

(51) Int. Cl. A61B 17/12

(52) U.S. Cl. 606/201

(57) ABSTRACT

A compression garment for use on a limb includes a stocking, a first fastening device, and a second fastening device. The stocking has upper and lower portions configured to apply compressive forces to the limb. The upper portion of the stocking is configured to at least partially wrap around the first fastening device during rotation thereof, and the lower portion of the stocking is configured to at least partially wrap around the second fastening device during rotation thereof whereby rotation of the first and second fastening devices adjusts compressive forces applied by the upper and lower portions of the stocking to the limb.

### BACKGROUND OF THE INVENTION

1. Field of the Invention

The present invention relates generally to garments used on bodily limbs for the treatment thereof, and more particularly, to garments which provide compressive forces to bodily limbs.

2. Description of Related Art

Arteries transport oxygen replenished blood from the heart to the rest of the body. Veins return oxygen depleted blood back to the heart. When the veins in the lower extremities of the body have difficulty transporting blood back to the heart, a condition develops called chronic venous insufficiency (CVI), also known as chronic venous disease (CVD). CVI most commonly occurs as the result of a blood clot in the deep veins of the legs, a disease known as deep vein thrombosis (DVT). CVI also results from pelvic tumors and vascular malformations, and sometimes occurs for unknown reasons. When a person is standing or sitting, blood in the veins of the legs flows in an upward direction. When the person walks, the calf muscles and muscles in the feet contract to squeeze the veins and push the blood upward. To keep the blood flowing upward and prevent it from flowing downward, the veins contain one-way valves. CVI occurs when these valves become damaged and allow the blood to leak back downward in the opposite direction. Such valve damage may occur as the result of aging, extended sitting or standing, or a combination of aging and reduced mobility. When the veins and valves become weakened and the blood does not properly flow up to the heart, blood pressure in the veins of the lower extremities can stay elevated for long periods of time, leading to CVI. This condition is more common in older individuals, and if not properly treated, can lead to burst capillaries, local tissue inflammation, internal tissue damage, varicose veins, ulcers, and open sores on the skin's surface.

CVI can diminish the capacity of the venous system and increase the workload of the lymphatic system in the affected area. The lymphatic system must then transport larger volumes of water and protein to reduce the fluid load in the affected tissues of the legs, a situation which is especially difficult for patients with Lymphedema, varicose veins, and other lower extremity pathology.

One non-surgical option often used to help prevent or treat the lower extremity pathologies discussed above is the use of compression stockings. Compression stockings help prevent leg fatigue, ankle and foot swelling, spider veins, and varicose veins. They improve circulation in the legs, especially when used in conjunction with frequent exercise and leg elevation. Compression stockings maintain pressure on the legs while allowing for normal ambulation. Increasing pressure in the tissues beneath the skin reduces excess leakage of fluid from the capillaries and increases absorption of tissue fluid by the capillaries and lymphatic vessels. In addition, the increased pressure decreases the size of the veins, which causes the blood to flow faster and help prevent it from pooling.

Compression stocking tightness typically varies between 15-50 mm HG. The tightness of a given stocking depends on its particular configuration and class. For example, stockings having a compression pressure of 15-20 mm HG are considered light compression stockings. Class I stockings are 20-30 mm Hg, class II stockings are 30-40 mm Hg, and class III stockings are 40-50 mm Hg.

While such compression stockings are a commonly utilized non-invasive treatment of lower leg pathology, the issues they present are numerous. Wearing a tightly fitting stocking can be tedious or time consuming to put on, and may require help from another person if the wearer is injured, elderly, or has some form of disability. In addition, the pressure applied by the stocking generally stays relatively constant during use without any option of increasing or decreasing the tightness level. As compression stockings are repeatedly worn, they lose elasticity and thus tightness over time. Once such prescribed elasticity and tightness is lost, the stocking is of little or no value, and needs to be replaced on account of its looseness, which requires buying a new pair to obtain the desired pressure.

### SUMMARY OF THE INVENTION

The subject invention is directed to a new and useful compression garment for use on a bodily limb. The compression garment includes a stocking configured and adapted to apply compressive forces about a limb, and a fastening device for adjusting the compressive forces applied by the stocking to the limb. The stocking preferably includes at least an upper portion and a lower portion. The compression garment may include one or more fastener assemblies for applying an adjustable compressive force to one or more stocking portions.

In certain embodiments, the stocking defines a channel for receivably engaging the fastener assembly, and the fastener assembly is rotatable within the channel in a first direction to increase compressive forces applied by the stocking to the limb, and a second direction to decrease compressive forces applied by the stocking to the limb. The channel preferably extends radially outward relative to the first section, and the fastener assembly is preferably insertable into the channel and removable therefrom.

In certain embodiments, each stocking portion includes an elastic first section and an elastic second section. The elastic first section is configured to at least partially wrap around the limb and apply compressive forces thereto, and has opposed first and second ends which define a gap therebetween. The elastic second section is coupled to the opposed first and second ends of the elastic first section and may have increased stretching properties relative to the elastic first section. The fastener assembly is operatively associated with the elastic second section of the stocking and configured to increase and decrease a size of the gap between the respective opposed first and second ends of the first section to adjust compressive forces applied by the stocking to the limb.

In accordance with certain embodiments, the second elastic section of the compression stocking is configured to wrap around the fastening device during rotation thereof in the first direction to tighten the compression stocking about the limb, and to unwrap from the fastening device during rotation thereof in the second direction opposite the first direction to loosen the compression stocking about the limb.

The compression garment of the present invention functions to provide compressive forces to the limb of a user, and is adjustable by the user during use. Such adjustability and ease of use facilitates faster changing time, reduces the need for assistance from another person, provides flexibility to a given garment in terms of the pressure levels it can provide, and increases the longevity of the garment for its intended purpose by allowing a user to compensate for lost elasticity after repeated use.

These and other features of the systems and methods of the subject invention will become more readily apparent to those skilled in the art from the following detailed description of the preferred embodiments taken in conjunction with the drawings.

### BRIEF DESCRIPTION OF THE DRAWINGS

So that those skilled in the art to which the subject invention appertains will readily understand how to make and use the devices and methods of the subject invention without undue experimentation, preferred embodiments thereof will be described in detail herein below with reference to certain figures, wherein:

FIG. 1 is an assembled perspective view of a first exemplary embodiment of a compression garment constructed in accordance with the present invention;

FIG. 2 is a partially exploded perspective rear view of the compression garment of FIG. 1;

FIG. 3 is a truncated perspective rear view of the compression garment of FIG. 1;

FIG. 4. is a truncated perspective rear view of the compression garment of FIG. 1 in a tightened configuration;

FIG. 5 is a top sectional view of the compression garment of FIG. 4;

FIG. 6 is a truncated perspective rear view of the compression garment of FIG. 4 with a strap of the upper portion in a closed position;

FIG. 7 is a top section view of the upper portion of the compression garment of FIG. 6;

FIG. 8 is an exploded perspective view of a second exemplary embodiment of a compression garment constructed in accordance with the present invention;

FIG. 9 is an exploded perspective view of one of the fastener assemblies of FIG. 8;

FIG. 10 is an assembled perspective view of the fastener assembly of FIG. 9;

FIG. 11 is a perspective truncated view of a first piece of one of the fastener assemblies of FIG. 8 being inserted into a channel of the stocking;

FIG. 12 is a perspective view of the first piece of the fastener assembly of FIG. 11 fully inserted into the channel of the stocking;

FIG. 13 is a perspective view of the first piece of the fastener assembly and stocking of FIG. 12 with the first piece rotated clockwise with the channel;

FIG. 14 is a top section view of the first piece of the fastener assembly and stocking of FIG. 13;

FIG. 15 is a perspective view of the first piece of the fastener assembly and stocking of FIG. 12 and a second piece of the fastener assembly shown in exploded view;

FIG. 16 is a perspective assembled view of the first and second pieces of the fastener assembly and the stocking of FIG. 15.

FIG. 17 is a top sectional view of the first and second pieces of the fastener assembly and the stocking of FIG. 16.

### DETAILED DESCRIPTION OF THE PREFERRED EMBODIMENTS

The present invention is now described more fully with reference to the accompanying drawings, in which an illustrated embodiment of the present invention is shown. The present invention is not limited in any way to the illustrated embodiment as the illustrated embodiment described below is merely exemplary of the invention, which can be embodied in various forms, as appreciated by one skilled in the art. Therefore, it is to be understood that any structural and functional details disclosed herein are not to be interpreted as limiting, but merely as a basis for the claims and as a representative for teaching one skilled in the art to variously employ the present invention. Furthermore, the terms and phrases used herein are not intended to be limiting but rather to provide an understandable description of the invention.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials are now described. All publications mentioned herein are incorporated herein by reference to disclose and describe the methods and/or materials in connection with which the publications are cited.

It must be noted that as used herein and in the appended claims, the singular forms “a”, “an,” and “the” include plural referents unless the context clearly dictates otherwise. Thus, for example, reference to “a stimulus” would include a plurality of such stimuli and reference to “the signal” would include reference to one or more signals and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may differ from the actual publication dates which may need to be independently confirmed.

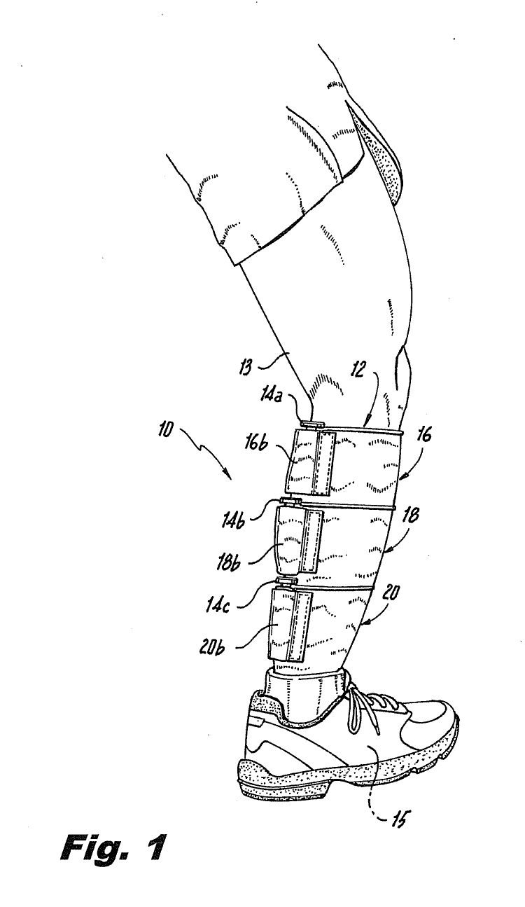

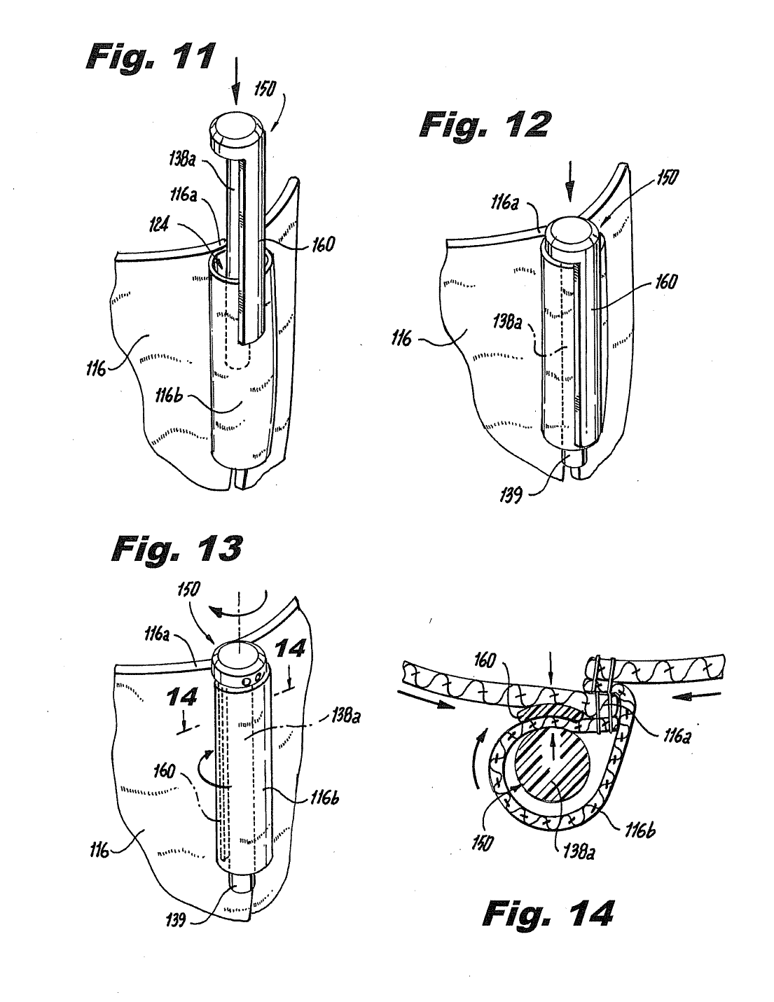

For purposes of explanation and illustration, and not limitation, a first exemplary embodiment of the compression garment, or aspects thereof, in accordance with the invention is shown in FIGS. 1-7 and is designated generally by reference character 10. A second exemplary embodiment in accordance with the invention, or aspects thereof, is shown in FIGS. 8-17 and is designated generally by reference character 100.

Turning now to FIGS. 1-2, the compression garment 10 includes a sleeve or stocking 12 which is sized and configured to receive the lower portion of a leg 13 of a person, and fastener assemblies 14a, 14b, 14c for adjusting compressive forces applied by the stocking 12 to the leg 13.

The stocking 12 is preferably configured with an integrally formed upper portion 16, an integrally formed central portion 18, and an integrally formed lower portion 20, all of which define a longitudinal axis 23 extending through the stocking 12, and each of which is independently adjustable by a respective fastener assembly (14a, 14b, 14c) to a particular tightness level (further discussed below). The stocking 12 may alternatively be constructed with the upper, central, and lower portions 16, 18, 20 formed separate, distinct, and detachable from one another (e.g., via a zipper, adhesive, interference fit, or other means), or with just one or two of the three portions 16, 18, 20.

As best shown in FIG. 2, the upper portion 16 of the stocking 12 defines an opening 22 leading to interior space defined by the upper, central, and lower portions 16, 18, 20 of the stocking 12. The upper portion 16 includes a first section 16a configured to at least partially wrap around the leg 13 and apply compressive forces thereto. The first section 16a extends between opposed first and second ends 17a, 17b which define a gap between them. The upper portion 16 also includes a second section 16b which extends between the opposed first and second ends 17a, 17b of the first section 16a, and radially outward relative to the first section 16a to define an upper channel 24 configured to receive the upper fastener assembly 14a.

The central portion 18 includes a first section 18a configured to at least partially wrap around the leg 13 and apply compressive forces thereto. The first section 18a extends between opposed first and second ends 19a, 19b which define a gap between them. The central portion 18 also includes a second section 18b which extends between the opposed first and second ends 19a, 19b of the first section 18a, and radially outward relative to the first section 18a to define a central channel 26 configured to receive the central fastener assembly 14b.

The lower portion 20 includes a first section 20a configured to at least partially wrap around the leg 13 and apply compressive forces thereto. The first section 20a extends between opposed first and second ends 21a, 21b which define a gap between them. The lower portion 20 also includes a second section 20b which extends between the opposed first and second ends 21a, 21b of the first section 20a, and radially outward relative to the first section 20a to define a lower channel 28 configured to receive the lower fastener assembly 14c. The lower portion 20 preferably also includes a foot portion 30 configured to receive the foot 15.

The stocking 12 preferably includes VELCRO® (e.g., loops & fasteners) flaps 32, 34, 36 for fixing the fastener assemblies 14a, 14b, 14c in place on the upper, central, and lower portions 16, 18, 20 to maintain a given tightness once the respective fastener assemblies have been adjusted.

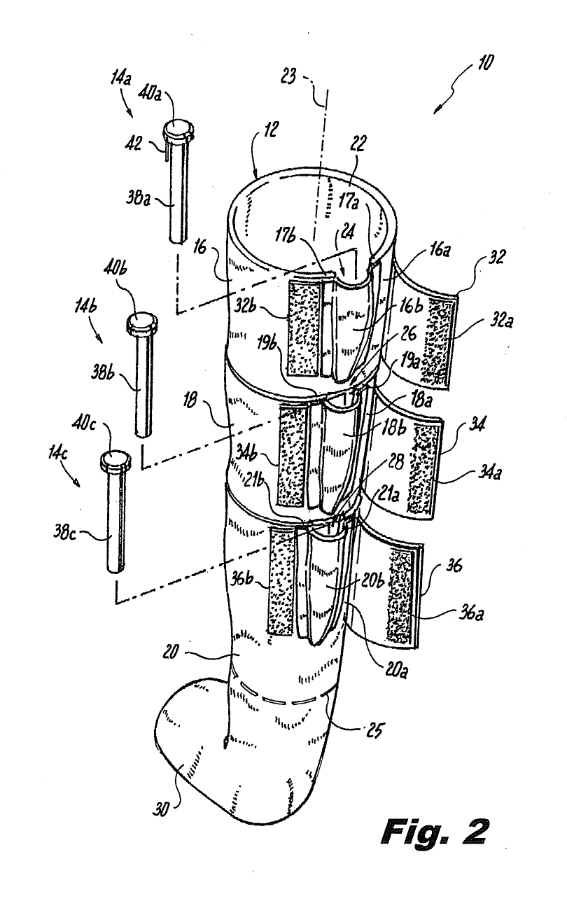

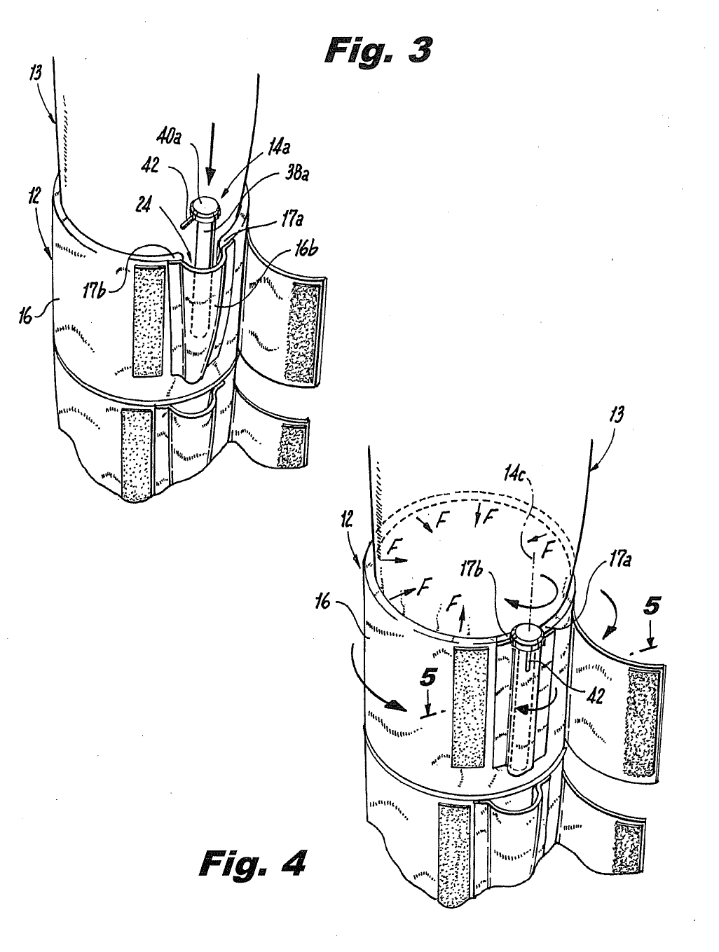

It will be appreciated that the stocking 12 is preferably constructed from flexible materials such as those commonly found in graduated compression hosiery or stockings which have a high elasticity suitable for stretching and maintaining compression on the leg, even after numerous uses. In addition, the second sections 16b, 18b, 20b of the upper, central, and lower portions 16, 18, 20 are preferably constructed from flexible materials which have a high elasticity suitable for stretching and maintaining compression on the leg after numerous uses. The second sections 16b, 18b, 20b may alternatively or additionally be constructed from materials which have increased stretching properties relative to the materials used for the first sections 16a, 18a, 20a to which they attach. The second sections 16b, 18b, 20b can stretch to wrap around the fastener assemblies 14a, 14b, 14c as they are adjusted during operation of the compression garment 10, further discussed below with respect to FIGS. 3-7.

Continuing with FIG. 2, the fastener assemblies 14a, 14b, 14c include elongated rods or plates 38a, 38b, 38c and corresponding heads 40a, 40b, 40c attached to the elongated rods 38a, 38b, 38c. The rods (e.g., rod 38a) may each optionally include a small catch member 42 for gripping an outer edge of the second section 16b of the upper portion 16 of the stocking 12 when the rod 38a of the fastener assembly 14a is inserted into the upper channel 24. The catch member 42 may be constructed to slidably engage and grip the second section 16b via an interference fit, or may be constructed as a small spring biased member which is upwardly movable away from the rod 38a when the rod 38a is inserted into the upper channel 24, and biased to snap back to pinch the second section 16b once the fastener assembly 14a is fully inserted into the channel 24.

Turning now to FIGS. 3-7, the setup and operation of the compression garment 10 is as follows. The stocking 12 is initially put on by a user in a straightforward manner. The foot 15 is inserted into the opening 22 of the upper portion 16 and pushed down and into the foot portion 30 while the stocking 12 is pulled up over the leg 13. As shown in FIGS. 3 and 4, the upper fastener assembly 14a is then inserted into the upper channel 24 until the elongated rod 38a is fully disposed therein with the head 40a disposed at the top thereof. The head 40a is then grasped in conjunction with the second section 16b which defines the channel 24. The optional catch member 42 is also pushed downward to pinch the second section 16b to the upper fastener assembly 14a, and the upper fastener assembly 14a is rotated in a clockwise direction with the second section 16b about a longitudinal axis 14c of the upper fastener assembly 14a as shown in FIGS. 4 & 5. The second section 16b stretches and wraps around the upper fastener assembly 14a as it is rotated, pulling the second end 17b inward toward the upper fastener assembly 14a as shown in FIGS. 4 & 5. It will be appreciated by those skilled in the art that the equal and opposite force exerted by the second section 16b on the first end 17a will cause the first end 17a to also move toward the upper fastener assembly 14a as shown in FIGS. 4 & 5, thus decreasing the size of the gap between the opposed first and second ends 17a, 17b of the first section 16a, whereby compressive forces applied by the stocking 12 to the leg 13 are adjusted. As a result, the upper portion 16 of the stocking 12 will tighten about the leg 13 and provide increased compressive forces F to the leg 13. The upper fastener assembly 14a may be rotated in the opposite, counter-clockwise direction to loosen the upper portion 16 about the leg 13 and decrease the compressive forces F provided thereto. It will also be appreciated that the upper fastener assembly 14a could alternatively be rotated from the position of FIG. 3 by initially grasping the head 40a and the first end 17a and rotating in a counter clockwise direction to tighten the upper portion 16 about the leg 13, and subsequently rotated in a clockwise direction to loosen it.

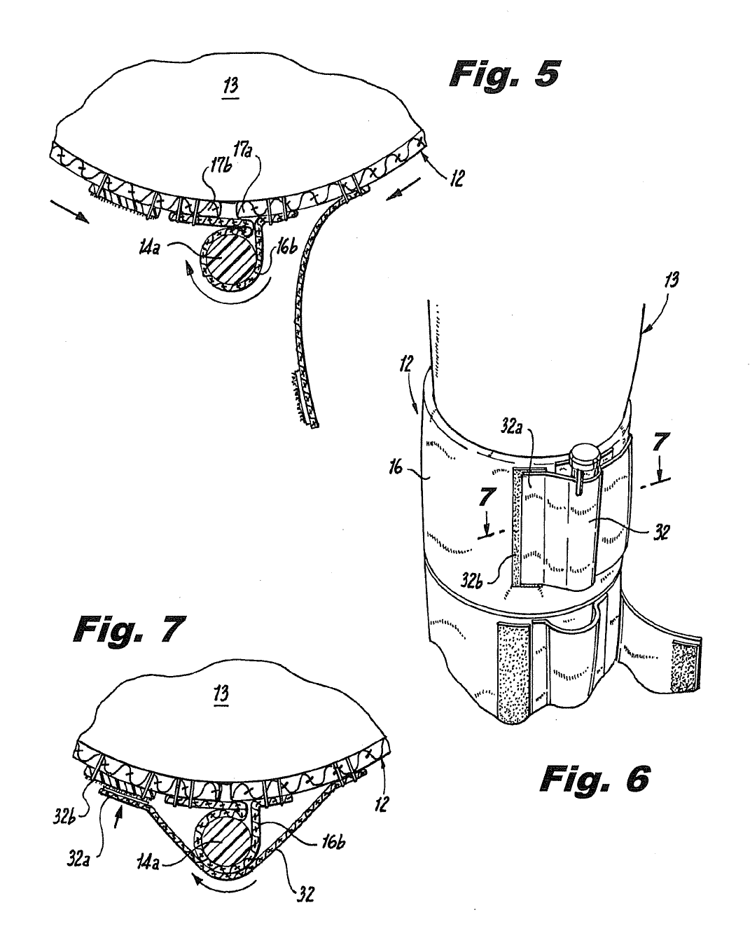

Once the desired tightness of the upper portion 16 about the leg 13 is achieved, the upper flap 32 is moved from the open position of FIGS. 2-5 to the closed position of FIGS. 6-7. The upper flap 32 is tightly secured over the upper fastener assembly 14a and respective loop and fastener sections 32a, 32b of the upper flap 32 are secured to one another as shown in FIGS. 6-7. The flap 32 is preferably secured tight enough to rotatably fix the upper fastener assembly 14a in position so as not to allow unwrapping of the second section 16b of the upper portion 16 from the upper fastener assembly 14a. It will be appreciated that the fastener sections 32a, 32b of the upper flap 32 can engage one another using any number of suitable attachment means, such as, by way of example, VELCRO® (e.g., loops & fasteners), one or more hooks corresponding to one or more clasps, etc.

The central and lower portions 18, 20 of the stocking 12 are tightened and loosened about the leg 13 in the same manner. The central fastener assembly 14b is inserted into the central channel 26 and rotated in a clockwise direction while grasping the second section 18b of the central portion 18 and the second end 19b of the first section 18a to tighten the central portion 18 about the leg 13, and rotated in a counter clockwise direction to loosen the central portion 18 about the leg 13. Once the desired tightness level of the central portion 18 is reached, the central flap 34 is tightly secured over the central fastener assembly 14b, and the respective loop and fastener sections 34a, 34b of the central flap 34 are secured to one another.

The lower fastener assembly 14c is inserted into the lower channel 28 and rotated in a clockwise direction while grasping the second section 20b of the lower portion 20 and the second end 21b of the first section 20a to tighten the lower portion 20 about the leg 13, and rotated in a counter clockwise direction to loosen the lower portion 20 about the leg 13. Once the desired tightness level of the lower portion 20 is reached, the lower flap 36 is tightly secured over the lower fastener assembly 14c, and the respective loop and fastener sections 36a, 36b of the lower flap 36 are secured to one another.

The upper, central, and lower portions 16, 18, 20 of the stocking 12 can each be adjusted during use, independent of one another, to tighten or loosen the respective portion of the stocking 12 about the leg 13. It will be appreciated that the central and lower portions 18, 20 could alternatively be rotated counterclockwise from the position of FIG. 3 for tightening thereof, and subsequently rotated in an opposite clockwise direction for loosening thereof as discussed above with respect to the upper portion 16 by simply grasping the first ends 19a, 21a of the respective first sections 18a, 20a when the central and lower fastener assemblies 14b, 14c are rotated counterclockwise from the initial position of FIG. 3.

It will also be appreciated that the stocking 12 can be utilized without the foot portion 30 by simply tearing or cutting the stocking 12 along a perforated portion 25 to create a new opening at the bottom thereof. Alternatively, the stocking 12 may simply be constructed without the foot portion 30, hence as a lower leg sleeve.

Turning now to FIGS. 8-17, a second exemplary embodiment of a compression garment in accordance with the invention, or aspects thereof, is designated generally by reference character 100. The compression garment 100 includes a stocking 112 sized and configured to at least partially wrap around the leg 13, and upper, central, and lower fastener assemblies 114a, 114b, 114c for adjusting compressive forces applied by the stocking 112 to the leg 13.

The stocking 112 includes an upper portion 116, a central portion 118, and a lower portion 120, all of which define a longitudinal axis 123 extending through the stocking 112, and each of which is independently adjustable by the respective upper, central, and lower fastener assembly 114a, 114b, 114c to a particular tightness level. The upper, central, and lower portions 116, 118, 120 define respective first sections 116a, 118a, 120a and respective second sections 116b, 118b, 120b. The second sections 116b, 118b, 120b define upper, central, and lower channels 124, 126, 128 extending radially outward from the first sections 116a, 118a, 120a relative to the longitudinal axis 123.

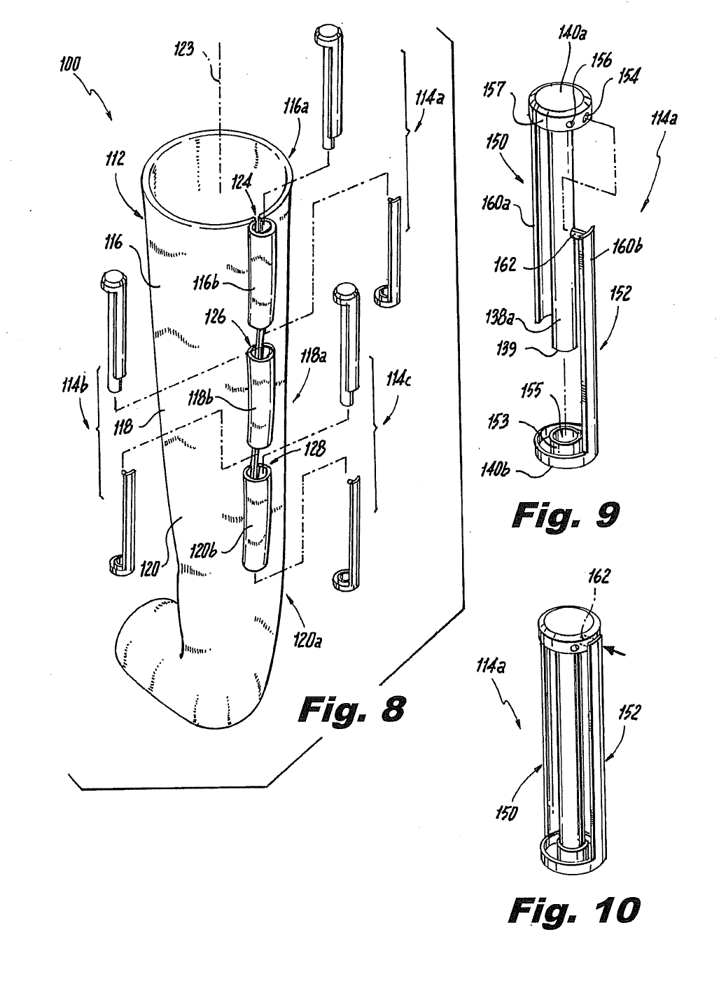

The fastener assemblies 114a, 114b, 114c are preferably formed identical to one another for ease of use and replacement. An illustrative embodiment of the upper fastener assembly 114a is most clearly shown in FIGS. 9 & 10. The upper fastener assembly 114a includes a first member 150 and a second member 152 which are configured to couple together about the second section 116b of the upper portion 116 to lock the upper portion 116 at a desired tightness as further discussed below with respect to FIGS. 11-17. The first member 150 has a head 140a, an elongated rod or plate 138a, and an elongated fin 160a. The head 140a of the first member 150 defines small holes 154, 156 extending through an outer surface 157 thereof. The second member 152 has a head 140b and an elongated fin 160b extending therefrom which defines a peg 162 at the end opposite the head 140b. The peg 162 is insertable into one of the holes 154, 156 via a press or interference fit. The head 140b of the second member 152 includes an annular wall 153 which defines an opening 155 sized to receive an end 139 of the elongated rod 138a, preferably via an interference fit. An assembled configuration of the upper fastener assembly 114a is shown in FIG. 10.

Turning now to FIGS. 11-17, the setup and operation of the compression garment 100 is similar to that described above with respect to FIGS. 3-7, except for the manner in which the fastener assemblies 114a, 114b, 114c are applied and utilized. As shown in FIGS. 11 & 12, the elongated rod 138a of the first member 150 of the upper fastener assembly 114a is inserted into the channel 124 with the fin 160 disposed outside thereof. The fin 160 is preferably formed in very close proximity to the elongated rod 138a so that the second section 116b which defines the channel 124 is press fit between the fin 160 and the elongated rod 138a. The wearer then grasps the first member 150 in conjunction with the second section 116b and rotates them together in a clockwise direction as illustrated in FIGS. 13 & 14. It will be appreciated that clockwise (or counterclockwise) rotation will pull one or both ends of the first section 116a toward and/or around the elongated rod 138a of the first member 150, covering the fin 160 as shown in FIGS. 13-15, thereby tightening the upper portion 116 of the stocking 112 about the leg.

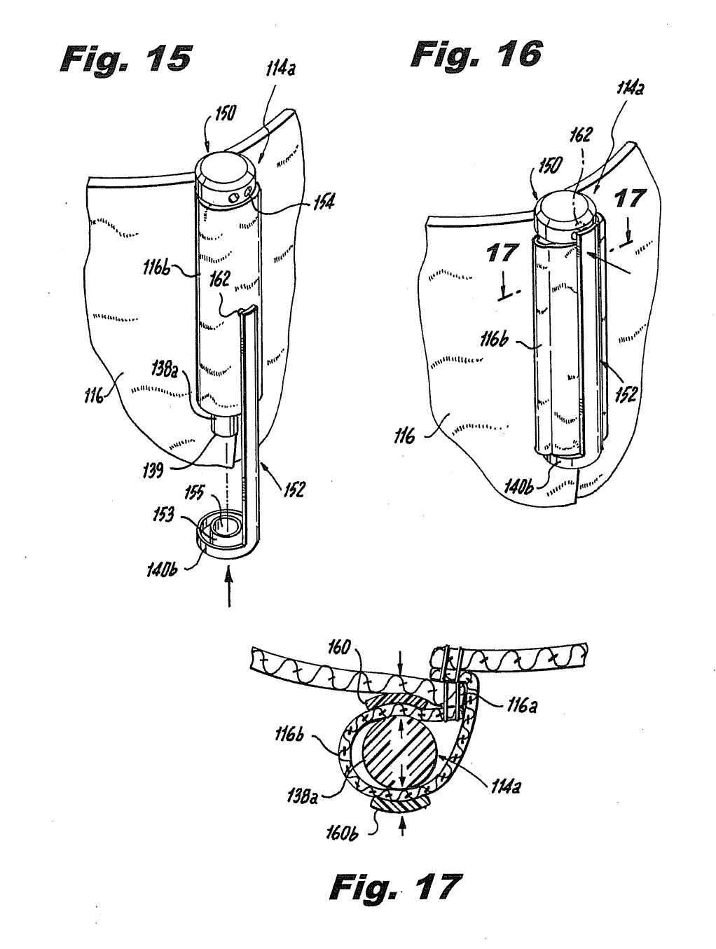

As shown in FIGS. 15-17, the second member 152 of the upper fastener assembly 114a is then coupled to the first member 150. The head 140b is pushed upward to receive the end 139 of the elongated rod 138a within the annular wall 153 via press fit. In addition, the fin 160b of the second member 152 is disposed outside of the second section 116b, and the peg 162 is inserted into the hole 154, thereby sandwiching the second section 116b between the fin 160b of the second member 152 and the elongated rod 138a of the first member 150 to prevent the second section 116b from unwrapping from the first member 150, and thus to limit or prevent movement or loosening thereof. The central and lower fastener assemblies 114b, 114c are similarly attached to the second sections 118b, 120b of the central and lower portions 118, 120 of the stocking 112.

The compression garment of the present invention functions to provide compressive forces to the limb of a user, and is adjustable by the user during use. Such adjustability and ease of use facilitates faster changing time, reduces the need for assistance from another person, provides flexibility to a given garment in terms of the pressure levels it can provide, and increases the longevity of the garment for its intended purpose by allowing a user to compensate for lost elasticity after repeated use.

The above presents a description of a best mode contemplated for carrying out the present invention, and of the manner and process of making and using the present invention, in such full, clear, concise, and exact terms as to enable any person skilled in the art to which it pertains to make and use these devices and methods. The present invention is, however, susceptible to modifications and alternative method steps from those discussed above that are fully equivalent. Consequently, the present invention is not limited to the particular embodiments disclosed. On the contrary, the present invention encompasses all modifications and alternative constructions and methods coming within the spirit and scope thereof.

The descriptions above and the accompanying drawings should be interpreted in the illustrative and not the limited sense. While the invention has been disclosed in connection with the preferred embodiment or embodiments thereof, it should be understood that there may be other embodiments which fall within the scope of the invention as defined by the following claims. Where a claim, if any, is expressed as a means or step for performing a specified function, it is intended that such claim be construed to cover the corresponding structure, material, or acts described in the specification and equivalents thereof, including both structural equivalents and equivalent structures, material-based equivalents and equivalent materials, and act-based equivalents and equivalent acts.

For example, while a compression stocking used on the leg of a person has been disclosed, it will be appreciated that the compression garment could also be formed as a sleeve used around an arm or other limb or body part of a person. While particular shapes, types, and respective sizes of fastener assemblies, securing flaps, upper, central, and lower portions of a compression garment have been disclosed, it will be appreciated that alternative shapes, types, and sizes of fastener assemblies, securing flaps, and upper, central, and lower portions of a compression garment may be utilized. It will therefore be appreciated that changes and/or modifications may be made without departing from the spirit and scope of the subject invention as claimed.

What is claimed is:

1. A compression garment for use on a limb, comprising:
(a) a sleeve configured and adapted to apply compressive forces to the limb; and
(b) at least a first fastening device configured to adjust the compressive forces applied by a first portion of the sleeve to the limb.

2. A compression garment as recited in claim 1, further including:
(c) a second fastening device configured to adjust compressive forces applied by a second portion of the sleeve to the limb.

3. A compression garment according to claim 2, wherein the first portion of the sleeve is configured to at least partially wrap around the first fastening device during rotation thereof, and the second portion of the sleeve is configured to at least partially wrap around the second fastening device during rotation thereof.

4. A compression garment according to claim 3, wherein the first and second fastening devices are rotatable in a first direction to increase compressive forces applied by the sleeve to the limb, and a second direction to decrease compressive forces applied by the sleeve to the limb.

5. A compression garment according to claim 1, wherein the limb is a leg and the sleeve is a stocking configured to receive the leg and apply compressive forces thereto.

6. A compression garment for use on a leg of a person, comprising:
(a) a stocking configured and adapted to receive the leg and apply compressive forces thereto; and
(b) a first fastening device integrated with the stocking for adjusting the compressive forces applied by the stocking to the leg,
wherein the stocking defines a first channel for receivably engaging the first fastening device, and the first fastening device is insertable into the first channel and rotatable in a first direction to increase compressive forces applied by the stocking to the leg, and a second direction to decrease compressive forces applied by the stocking to the leg.

7. A compression garment according to claim 6, wherein the fastening device includes a first member and a second member, the stocking is configured to at least partially wrap around the first member of the fastening device during rotation thereof, and the first and second members are configured to couple to one another to sandwich a partially wrapped portion of the stocking therebetween.

8. A compression garment according to claim 6, wherein the stocking defines a longitudinal axis, and the channel extends radially outward relative to the longitudinal axis.

9. A compression garment according to claim 6, further comprising:
(c) a second fastening device integrated with the stocking for adjusting compressive forces applied by the stocking to the leg.

10. A compression garment according to claim 9, wherein the stocking defines a second channel for receivably engaging the second fastening device, and the second fastening device is insertable into the second channel and rotatable in a first direction to increase compressive forces applied by the stocking to the leg, and a second direction to decrease compressive forces applied by the stocking to the leg.

11. A compression garment for use on a limb, comprising:
(a) a first sleeve having an elastic first section and an elastic second section, the elastic first section configured to at least partially wrap around the limb and apply compressive forces thereto, the elastic first section having opposed first and second ends which define a gap therebetween, the elastic second section coupled to the opposed first and second ends of the elastic first section and having increased stretching properties relative to the elastic first section; and
(b) a first fastening device operatively associated with the elastic second section of the first sleeve and configured to increase and decrease a size of the gap between the opposed first and second ends of the elastic first section to adjust compressive forces applied by the sleeve to the limb.

12. A compression garment according to claim 11, wherein the second section is configured to wrap around the fastening device during rotation thereof in a first direction to tighten the first sleeve about the limb, and to unwrap from the fastening device during rotation thereof in a second direction to loosen the first sleeve about the limb.

13. A compression garment according to claim 12, wherein the second direction is opposite the first direction.

14. A compression garment according to claim 11, further comprising:
(c) a second sleeve having an elastic third section and an elastic fourth section, the elastic third section configured to at least partially wrap around the limb and apply compressive forces thereto, the elastic third section having opposed first and second ends which define a gap therebetween, the elastic fourth section coupled to the opposed first and second ends of the elastic third section and having increased stretching properties relative to the elastic third section.

15. A compression garment according to claim 14, further comprising:
(d) a second fastening device operatively associated with the elastic fourth section of the second sleeve and configured to increase and decrease a size of the gap between the opposed first and second ends of the elastic third section to adjust compressive forces applied by the second sleeve to the limb.

16. A compression garment according to claim 15, wherein the first and second sleeves are integrally formed with one another.
